(19) United States
(12) Patent Application Publication
(10) Pub. No.: US 2015/0301047 A1
(43) Pub. Date: Oct. 22, 2015

(54) METHOD FOR RECOGNITION OF A PRESENCE OF AN INFECTION WITH CMV

(71) Applicant: RED FLAG DIAGNOSTICS GMBH, Saarbrücken (DE)

(72) Inventors: Ferdinand Hermann Bahlmann, Hannover (DE); Danilo Fliser, Heidelberg (DE); Martina Sester, Homburg (DE); Urban Sester, Homberg (DE)

(21) Appl. No.: 14/646,767

(22) PCT Filed: Nov. 25, 2013
(86) PCT No.: PCT/EP2013/074603

§ 371 Date: May 22, 2015

Related U.S. Application Data
(60) Provisional application No. 61/729,473, filed on Nov. 23, 2012.

(30) Foreign Application Priority Data
Nov. 23, 2013 (EP) 12194069.6

(51) Int. Cl. G01N 33/569

(52) U.S. Cl. CPC G01N 33/56994; G01N 2333/045

(57) ABSTRACT

A method for recognition of a presence of an infection with CMV in a patient by—determination of a level of CD28neg/CD27neg CD4 T-cells in a whole blood sample obtained from the patient;—comparing the level of CD28neg/CD27neg CD4 T-cells with a level of a control group not infected with CMV; and—defining the presence of the infection with CMV, if the level of CD28neg/CD27neg CD4 T-cells in the patient is significantly higher than the level of CD28neg/CD27neg CD4 T-cells of the control group.

The present invention pertains to a method for recognition of a presence of an infection with CMV.

The cytomegalovirus (CMV) also named human cytomegalovirus (HCMV) or herpes virus 5 (HHV 5) causes numerous infectious diseases, further references to http://en.wikipedia.org/wiki/Human_cytomegalovirus.

The virus persists lifelong in the human cells of an infected individual. Even after termination of the disease it can be excreted intermittently without symptoms via body fluids for the rest of the person's life. The first infection with HCMV frequently shows little or none severe symptoms so that the infected individual does not even realise that he or she is infected. In rare cases, it can be associated with mononucleosis-like syndromes including prolonged fever and cervical adenopathy. Moreover, primary infections in immunocompetent individuals may rarely be accompanied by arthralgia and arthritis, colitis, pneumonitis, hepatitis or myocarditis. Up to 60% of the healthy population carries the HCMV which persists lifelong in lymphatic tissue.

In contrast, CMV infections are significant in the perinatal period and in immuno-compromised individuals. Among these individuals are patients with transplanted organs, transplantated stem cells, treated with cytostatic agents, or individuals suffering from AIDS which have a low CD4+ level. CMV-related disease symptoms are far more common in immunocompromised patients after organ or stem-cell transplantation, where long-term immunosuppression can lead to uncontrolled viral replication and serious disease. Beside the level of immunosuppressive drugsm the risk is largely determined by the CMV-infection status of the donor and recipient. The risk for CMV-related complications is lowest in CMV-negative recipients of a negative graft. It is moderately increased if the recipient is CMV-positive, whereas it is highest if CMV-negative patients receive a graft from a CMV-positive donor. Under these circumstances CMV infection will lead in almost all organs to interstitial infections of lymphocytes or plasma cells showing giant cells in the nucleus or cytoplasma. Pneumonia, retinitis, colitis and hepatitis are occurring frequently; further bacterial super-infections may occur which can lead to lethal complications.

There is a discrimination between a CMV disease showing clinical symptoms of the patient and a CMV infection, which can only be realised after diagnostic investigations (detection of the CMV in blood by CMV-PCR or pp65 antigen). Unfortunately these diagnostic tests are not optimal, for example CMV-PCR shows in 25% false-positive results.

Sester et al. reports in Transplantation 2001 (Sester M, Sester U, Gartner B, Heine G, Girndt M, et al. (2001) Levels of virus-specific CD4 T cells correlate with cytomegalovirus control and predict virus-induced disease after renal transplantation. Transplantation 71: 1287-1294) and AJT 2005 (Sester U, Gartner BC, Wilkens H, Schwaab B, Wossner R, et al. (2005) Differences in CMV-specific T-cell levels and long-term susceptibility to CMV infection after kidney, heart and lung transplantation. Am J Transplant 5: 1483-1489.) about an improved detection method, however, the method is unfavourable because it is long lasting—about 8 hours before a result is obtained—and furthermore the T-cells have to be functionally intact which leads to a laborious treatment of the sample to be analysed for example permanent cooling about 4° C. (2° C.-6° C.) and performing the measurement within 24 hours after taking the sample.

Jesser et al. 2006 (Jesser RD, Li S, Weinberg A (2006) Regulatory T cells generated during cytomegalovirus in vitro stimulation of mononuclear cells from HIV-infected individuals on HAART correlate with decreased lymphocyte proliferation. Virology 352: 408-417.) and Tovar-Salazar et al 2010 (Tovar-Salazar A, Patterson-Bartlett J, Jesser R, Weinberg A (2010) Regulatory function of cytomegalovirus-specific CD4+CD27-CD28- T cells. Virology 398: 158-167) have shown that the frequency of CMV specific T-cells (defined as CD27neg CD28neg CD4 T-cells) is in reciprocal correlation to in vitro CMV specific proliferation of PBMNCs (Jesser 2006) and that these express a higher amount of PD1 (Tovar-Salazar et al 2010).

One object of the present invention is providing a method which avoids the drawbacks of the prior art.

The object is accomplished by a method for recognition of a presence of an infection with CMV in a patient by
determination of a level of CD28neg/CD27neg CD4 T-cells in a whole blood sample obtained from the patient;
comparing the level of CD28neg/CD27neg CD4 T-cells with a level of a control group not infected with CMV; and
defining the presence of the infection with CMV, if the level of CD28neg/CD27neg CD4 T-cells in the patient is significantly higher than the level of CD28neg/CD27neg CD4 T-cells of the control group

The method of the invention is based on the surprising finding that a significant amount of the CD28neg/CD27neg/CD4 T-cells are specific for CMV. The CD28neg/CD27neg/CD4 T-cells can be used therefore as surrogates in the assay described by Sester et al.

The method of the invention can be used for differentiation between an inactive or active CMV infection. In case of transplantation, an active (primary) CMV-infection can be detected by measuring PD-1 or CTLA-4 expression of CD28neg/CD27neg CD4 T-cells and if the expression increases over time is an indication of a viraemia. Surprisingly, only the measuring a PD-1 expression and not CTLA-4 expression on CD28neg/CD27neg CD4 T-cells can identify a reactivation of CMV, if the expression increases over time is an indication of a viraemia.

The method of the invention can be used for a risk assessment for complications caused by a CMV infection in a transplant recipient.

In a further embodiment the method of invention can be used for monitoring progress of the treatment of CMV with antiviral medicaments.

According to the invention the level of CD28neg/CD27neg CD4 T-cells is determined in particular by flow cytometry.

In yet an embodiment of the invention the level of CD28neg/CD27neg CD4 T-cells is determined by a 4 colour analysis with antibodies against CD4, CD27, CD28, and PD-1 which are each differently labelled.

In still another embodiment of the invention the blood sample is treated by mixing with antibodies against CD28, CDs 27, CD4 and PD-1, followed by an incubation at ambient temperature, addition of a lysing solution followed by an incubation, separation of the cells, and performing a flow cytometry.

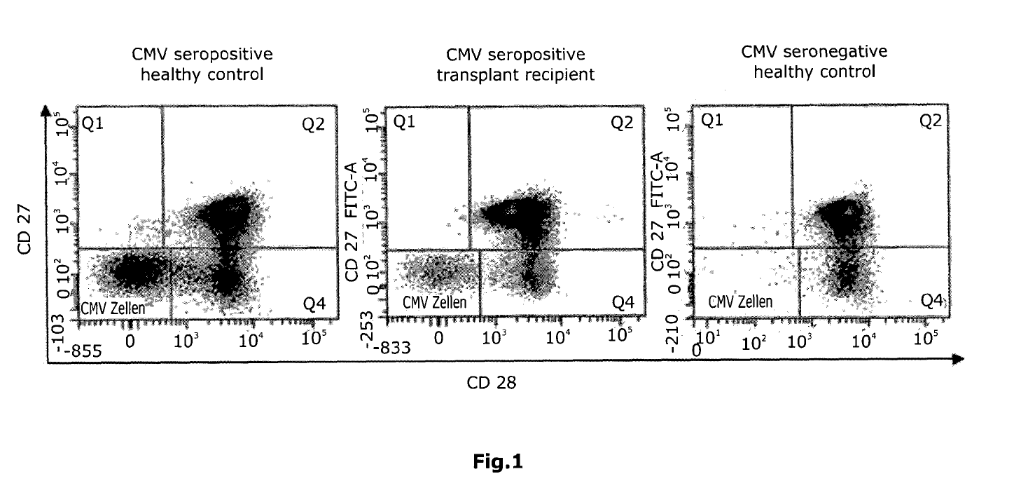

FIG. 1: Population of CD28neg/CD27neg/CD4 T-cells CMV sero-positivity.

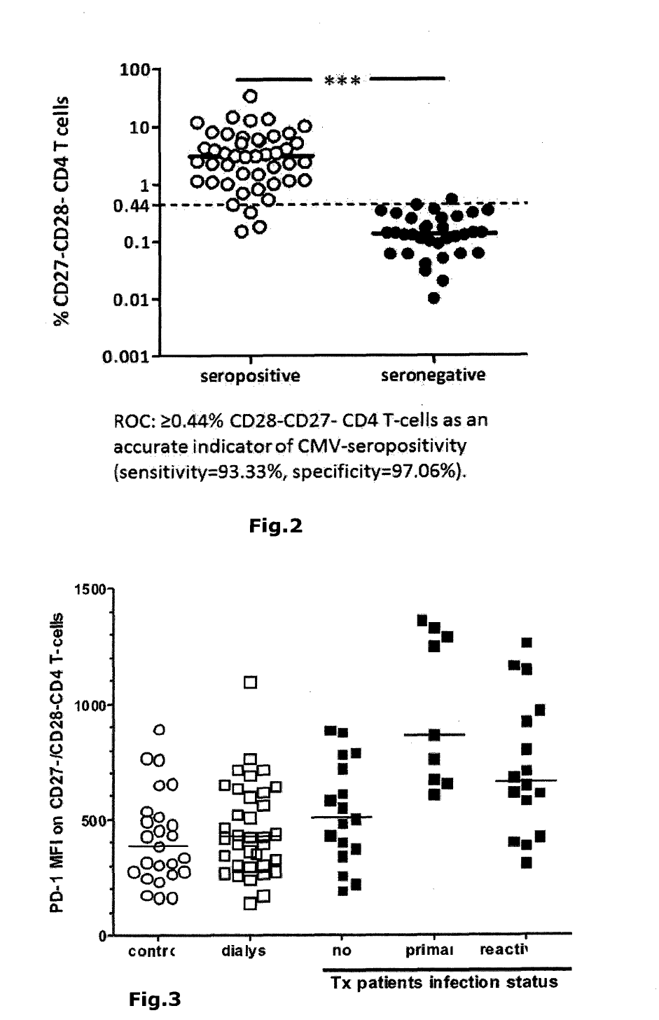

FIG. 2: Percentage of ≧0.44 CD28neg/CD27neg/CD4 T-cells discriminates between CMV-sero-positivity and sero-negativity.

FIG. 3: PD-1 on CD28-CD27 CD4 T-Cells is upregulated during both primary CMV infection and reactivation

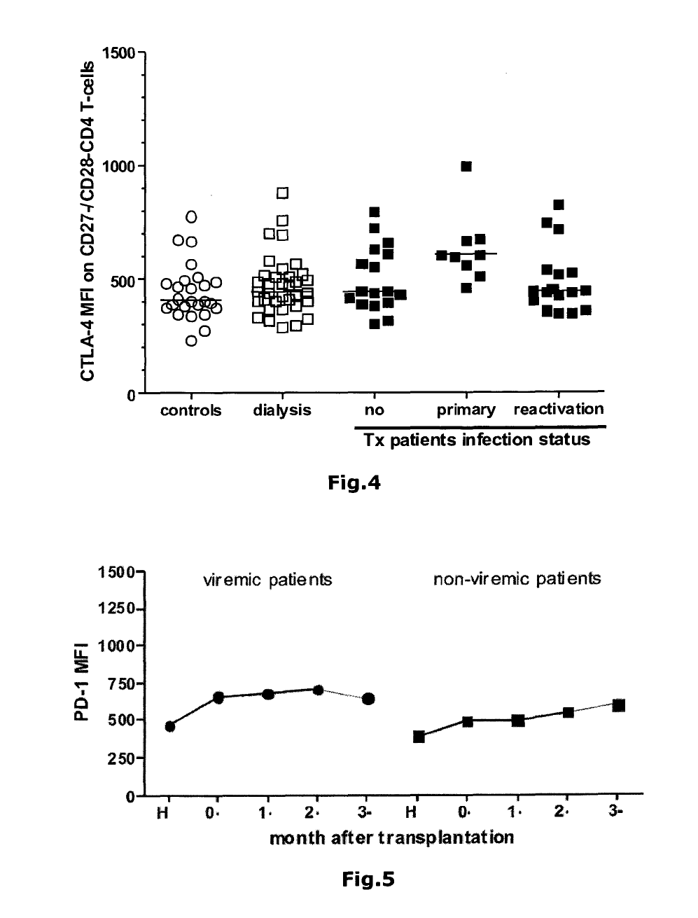

FIG. 4: CTLA-4 on CD28-CD27 CD4 T-Cells is upregulated during primary CMV infection but not reactivation

FIG. 5: Longitudinal analysis of PD1 MFI on CD28-CD27-CD4 T cells after transplantation

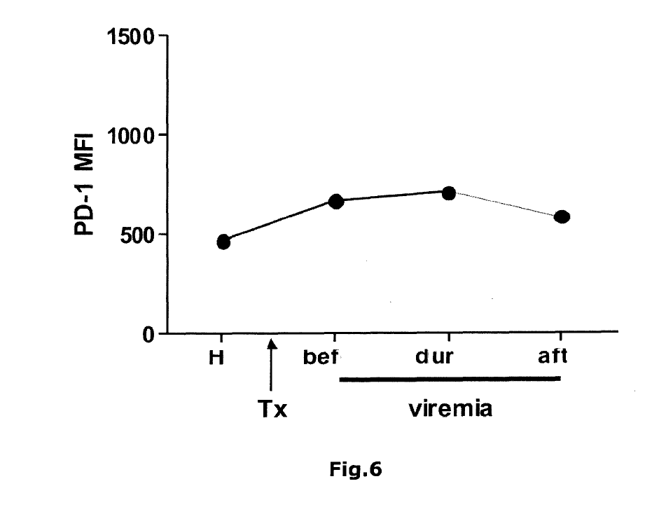

FIG. 6: Longitudinal analysis of PD1 MFI on CD28-CD27-CD4 T cells in relation to viremia

The method of the present invention provides for an assay starting from whole blood; determination of an cut off value to discriminate between seropositive or seronegative patients, for example 0.61% CD28neg CD27neg CD4 T-cells at the sensitivity of 88.89% and a specificity of 100% or ≧44% CD28neg CD27neg CD4 T-cells at the sensitivity of =93.33% and a specificity of =97.06% (FIGS. 1 and 2). A control is for example represented by a ROC-analysis. An example as shown in FIG. 2 has been performed with 45 CMV-seropositive and 34 CMV-seronegative healthy controls which in this case identified a percentage ≧44% CD28-/CD27-CD4 T-cells as an accurate indicator of CMV-seropositivity (sensitivity=93.33%, specificity=97.06%). A cut-off value is 0.61% CD28neg CD27neg CD4 T-Cells at a speciticity of 100% and a sensitivity of 88,89%.

A viraemia can be predicted by means of the increase of the expression of PD-1 (FIG. 3). This enables the medical practitioner to specifically initiate anti-viral treatment prior to the first appearance of initial symptoms of an active CMV infection. The PD-1 expression of the CD27neg CD4 T-cells increases from a mean value (MFI: 527) prior to transplantation operation to higher values (MFI: 722) closely prior to the onset of a viraemia or from that value (MFI: 722) to a still higher value of (MFI: 803) in the viraemia. S

FIG. 4 shows results obtained by monitoring the CTLA-4 on CD28neg CD27neg CD4 T-cells. In this case the CTLA-4 on CD28neg CD27neg CD4 T-cells is upreguleted during primary CMV infection but not in the case of reactivation.

The graph of the left part of FIG. 5 shows the course of an infection. The right part of FIG. 3 shows the graph of non-viraemic patients. From the slope of the left graph which is steeper than that of the non-viraemic patients the medical practitioner learns that an infection caused by CMV starts so that a therapy has to be initiated. When the slope levels off the practitioner knows that the treatment is effective. FIG. 6 summarizes this result.

The method of the invention provides the following advantages:
it allows stimulation-independent identification of CMV-associated cellular immunity;
it is more rapid, <1.5 h versus about 8 h duration of the conventional test;
it is easier, simple surface staining, only 100 μl blood is needed, no incubation period;
better logistics, surface staining allows for longer storing of samples compared to stimulation based assays.

The method of the invention is further described by the following protocol.

Antibodies against CD27, CD28, CD4 and PD1 (CTLA-4) were added in titrated amounts to the 100 μl of whole blood, mixed and incubated for 20 min at room temperature in the dark. Subsequently, the erythrocytes were lysed, washed ones with FACS buffer consisting of phosphate buffered saline, 5% filtered fetal calf serum, 0.5% bovine serum albumin and 0.07% sodium azide and thereafter the expression of the antigens on the cells were analyzed.

1. A method for identifying the presence of an infection with CMV in a patient comprising:
a) determining a level of CD28neg/CD27neg CD4 T-cells in a whole blood sample obtained from the patient;
b) comparing the level of CD28neg/CD27neg CD4 T-cells with a level of a control group not infected with CMV; and
c) defining the presence of the infection with CMV if when the level of CD28neg/CD27neg CD4 T-cells in the patient is significantly higher than the level of CD28neg/CD27neg CD4 T-cells of the control group.

2. The method of claim 1, further comprising differentiating between an inactive or active CMV infection by measuring a PD-1 expression of CD28neg/CD27neg CD4 T-cells, wherein expression increasing over time is an indication of a viraemia.

3. The method claim 1, wherein said patient is an immune incompetent patient, transplant recipient or newborn.

4. The method of claim 1, further comprising performing steps a)-c) a second time following treatment of said patient with an anti-CMV antiviral medicaments.

5. The method of claim 1, wherein the level of CD28neg/CD27neg CD4 T-cells is determined by flow cytometry.

6. The method of claim 5, wherein the level of CD28neg/CD27neg CD4 T-cells is determined by a multicolor color analysis with antibodies against CD4, CD27, CD28, and PD-1 which are each differently labelled.

7. The method of claim 1, wherein the blood sample is treated by mixing with antibodies against CD28, CDs 27, CD4 and PD-1, followed by an incubation at ambient temperature, addition of a lysing solution, followed by an incubation, separation of the cells, and performing flow cytometry.

8. The method of claim 2, wherein the level of CD28neg/CD27neg CD4 T-cells is determined by flow cytometry.

9. The method of claim 3, wherein the level of CD28neg/CD27neg CD4 T-cells is determined by flow cytometry.

10. The method of claim 4, wherein the level of CD28neg/CD27neg CD4 T-cells is determined by flow cytometry.

11. The method of claim 8, wherein the level of CD28neg/CD27neg CD4 T-cells is determined by a multicolor color analysis with antibodies against CD4, CD27, CD28, and PD-1 which are each differently labelled.

12. The method of claim 9, wherein the level of CD28neg/CD27neg CD4 T-cells is determined by a multicolor color analysis with antibodies against CD4, CD27, CD28, and PD-1 which are each differently labelled.

13. The method of claim 10, wherein the level of CD28neg/CD27neg CD4 T-cells is determined by a multicolor color analysis with antibodies against CD4, CD27, CD28, and PD-1 which are each differently labelled.

14. The method of claim 2, wherein the blood sample is treated by mixing with antibodies against CD28, CDs 27, CD4 and PD-1, followed by an incubation at ambient temperature, addition of a lysing solution, followed by an incubation, separation of the cells, and performing flow cytometry.

15. The method of claim 3, wherein the blood sample is treated by mixing with antibodies against CD28, CDs 27, CD4 and PD-1, followed by an incubation at ambient temperature, addition of a lysing solution, followed by an incubation, separation of the cells, and performing flow cytometry.

16. The method of claim 4, wherein the blood sample is treated by mixing with antibodies against CD28, CDs 27, CD4 and PD-1, followed by an incubation at ambient temperature, addition of a lysing solution, followed by an incubation, separation of the cells, and performing flow cytometry.

17. The method of claim 5, wherein the blood sample is treated by mixing with antibodies against CD28, CDs 27, CD4 and PD-1, followed by an incubation at ambient temperature, addition of a lysing solution, followed by an incubation, separation of the cells, and performing flow cytometry.

18. The method of claim 6, wherein the blood sample is treated by mixing with antibodies against CD28, CDs 27, CD4 and PD-1, followed by an incubation at ambient temperature, addition of a lysing solution, followed by an incubation, separation of the cells, and performing flow cytometry.
